(19) United States
(12) Patent Application Publication
(10) Pub. No.: US 2008/0269354 A1
(43) Pub. Date: Oct. 30, 2008

(54) COMPOSITION CONTAINING AN OILY CONTINUOUS PHASE

(75) Inventors: Euriel Clavel, Paris (FR); Pascal Arnaud, L'Hay Les Roses (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/062,640

(22) Filed: Apr. 4, 2008

Related U.S. Application Data
(60) Provisional application No. 60/980,177, filed on Oct. 16, 2007.

(30) Foreign Application Priority Data
Apr. 13, 2007 (FR) 07 54460

(51) Int. Cl. A61K 47/30; B01F 3/12; B01F 3/08

(52) U.S. Cl. 514/772.3; 516/31; 516/22

(57) ABSTRACT

Composition containing an oily continuous phase and a particulate phase, the oily continuous phase containing at least one dipentaerythritol ester, the particulate phase containing particles of acrylic polymer. Process for making up or caring for the skin, comprising the application of the invention composition to the skin.

### REFERENCE TO PRIOR APPLICATIONS

This application claims priority to U.S. provisional application 60/980,177 filed Oct. 16, 2007, and to French patent application 0754460 filed Apr. 13, 2007, both incorporated herein by reference.

### FIELD OF THE INVENTION

The present invention relates to a composition comprising an oily continuous phase, fillers and specific oils. Preferably, the invention compositions are useful for making up or caring for the skin. The invention also relates to a process for making up or caring for human skin, comprising the application of the invention compositions to the skin.

The invention compositions may be in any form, including that of a foundation, an eye shadow, a face powder, a concealer product, and a body make-up product. More especially, the invention relates to a foundation composition. In this regard, the invention compositions are preferably used for making up the skin.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Foundation compositions are commonly used to give an aesthetic colour to the skin, especially to the face, but also to hide skin imperfections such as redness or marks, or else to confer a matte appearance on the skin by absorption of sebum.

To do this, they contain a pulverulent phase made up of pigments and fillers. The greater the content of pigments and fillers in the composition, the more successfully the skin colour defects will be hidden, or the more successfully the sebum will be absorbed. However, too high a content of pigments and fillers often leads to keratin materials drying out, thereby causing the user to have a feeling of tautness, thus making the make-up uncomfortable to wear during the day.

It is known practice to introduce, into cosmetic compositions, oils or fatty substances in order to reduce the feeling of drying out on application and to improve the comfort of the make-up. However, the introduction of liquid or pasty fatty substances into make-up compositions has the drawback of leaving a greasy feeling on the skin, or even a tacky feel, which makes them relatively unattractive to users.

### SUMMARY OF THE INVENTION

There therefore exists a need for compositions useful for making up or caring for the skin which make it possible to satisfactorily hide the colour and/or relief imperfections of the skin, and to obtain good absorption of sebum, without however drying out the skin or, conversely, giving the user a greasy or even tacky feeling on application or during the day.

The inventors have discovered that it is possible to provide such a composition by combining particles of poly(methyl methacrylate) and dipentaerythritol ester oil in a composition comprising an oily continuous phase. The compositions obtained have, moreover, beneficial properties of being soft and easy to spread when they are applied to the skin.

### DETAILED DESCRIPTION OF THE PREFERRED EMBODIMENTS

According to a first aspect, a subject of the present invention is a composition comprising an oily continuous phase and a particulate phase, said oily continuous phase comprising at least one dipentaerythritol ester, and said particulate phase comprising particles of acrylic polymer.

According to a second aspect, a subject of the present invention is a process for making up and/or caring for the skin, comprising the application to said skin of a composition as described above.

According to a third aspect, a subject of the invention is also the use of a composition as described above, for producing a make-up that allows good absorption of sebum, without drying out the skin.

Yet another subject of the invention is the use of a composition as described above, for producing a make-up that is comfortable for the user and does not give the latter a greasy or even tacky feeling on application or during the day.

### Particles of Acrylic Polymer(s)

The composition according to the invention comprises particles of acrylic polymers.

The term “particles of acrylic polymer” means fillers obtained by polymerization of an alkyl acrylate and/or methacrylate monomer(s), in the form of a homopolymer or of a copolymer. By way of nonlimiting illustration, mention may in particular be made of particles of polymethyl methacrylate, of polymethyl methacrylate/ethylene glycol dimethacrylate, of polyallyl methacrylate/ethylene glycol dimethacrylate and of ethylene glycol dimethacrylate/lauryl methacrylate copolymer.

In particular, these particles are not film-forming, i.e. they do not form a continuous film when they are deposited on a support such as the skin.

The particles of acrylic polymers are generally preferably in the form of white-coloured, hollow or solid, spherical particles of which the number-average size is generally preferably on the micrometer scale, in particular ranges from 3 to 20 microns, and generally ranges from 7 to 15 microns, including 4, 5, 6, 7, 8, 9, 10, 11, 12, 13, 14, 15, 16, 17, 18, and 19 microns, and all subranges and values between stated values. The term “number-average size” denotes the dimension given by the random particle size distribution to half the population, known as D50.

By way of nonlimiting representation of the particles of acrylic polymers suitable for the invention, mention may in particular be made of:

particles of crosslinked polymethyl methacrylate, for instance “Covabead LH85” sold by the company LC Wackherr, or of noncrosslinked polymethyl methacrylate, such as SJ Touch 1 sold by the company Nihon Junyaku,

the particles of methyl methacrylate/butyl acrylate copolymer sold under the name Sepipress M by the company Seppic,

the particles of methyl acrylate/ethylene copolymer sold under the name EMAA by the company Kobo Products Inc.,

the particles of methyl methacrylate/ethylene glycol dimethacrylate crosslinked copolymer sold under the name Ganzpearl GMP 0820 by the company Ganz Chemical under the name Techpolymer MBP-8 by the company Sekisui Plastics, or else under the name SUNPMMA-S by the company Sunjin Chemical,

particles of polymethyl methacrylate/ethylene glycol dimethacrylate, for example “Dow Corning 5640 Microsponge Skin Oil Adsorber” sold by the company Dow Corning,

the particles of methyl methacrylate/ethylene glycol dimethacrylate crosslinked copolymer sold under the name Ganzpearl PM 030 by the company Ganz Chemical,

particles of polyallyl methacrylate/ethylene glycol dimethacrylate, for instance “Poly-Pore L200” or “Poly-Pore E200” sold by the company Amcol,

particles of lauryl methacrylate/ethylene glycol dimethacrylate copolymer, for instance “Polytrap 6603” sold by the company Dow Corning.

The amount of the particles of acrylic polymer(s) according to the invention is not particularly limited, for example, they may be present in the composition in an amount ranging from 0.1% to 10% by weight, relative to the total weight of the composition, in particular from 2% to 7%, and more particularly from 3% to 6% by weight.

In the context of the present invention, the particles of acrylic polymers are advantageously formulated in the oily continuous phase of the composition according to the invention.

### Dipentaerythritol Esters

The oily continuous phase of the composition according to the invention comprises at least one dipentaerythritol ester, this ester not constituting an “oil” in the oily phase as used herein.

The dipentaerythritol esters according to the invention are preferably dipentaerythritol C₃₋₂₄esters that may be obtained, e.g., by complete esterification of dipentaerythritol with linear or branched, saturated or unsaturated acids containing from 3 to 24 carbon atoms. Preferably, the dipentaerythritol C₃₋₂₄esters have from 4 to 18 carbon atoms in the ester chain (i.e., dipentaerythritol C₄₋₁₈esters), and even more preferably from 5 to 16 carbon atoms (i.e., dipentaerythritol C₅₋₁₆esters).

The term “complete esterification” is intended to mean the complete absence of any residual hydroxyl function.

The acids used to esterify the dipentaerythritol may be used in the form of a mixture.

The esters of the invention are in liquid form at ambient temperature (25° C.).

In a nonlimiting manner, mention may, by way of example of the dipentaerythritol esters used according to the present invention, be made of:

the dipentaerythrityl hexacaprylate/caprate sold under the name DUB DPHCC by the company Stearinerie Dubois,

the dipentaerythrityl hexa C₅₋₉ carboxylate sold under the name LexFeel 350 by the company Inolex Chemical Company.

According to a preferred embodiment, the preferred dipentaerythritol ester is dipentaerythrityl hexacaprylate/caprate.

The dipentaerythritol esters may be present in the composition in an amount ranging from 0.1% to 30% by weight, relative to the total weight of the composition, in particular from 1% to 20%, and more particularly from 3% to 15% by weight.

oily Phase

In addition to the dipentaerythritol ester, the oily phase of the composition according to the invention may comprise at least one additional volatile or non-volatile oil.

The term “volatile oil” means an oil capable of evaporating from the skin in less than one hour at ambient temperature and atmospheric pressure. This oil has in particular a vapour pressure, at ambient temperature (25° C.) and atmospheric pressure (760 mmHg) ranging from 0.13 Pa to 40 000 Pa (10⁻³ to 300 mmHg), especially ranging from 1.3 Pa to 13 000 Pa (0.01 to 100 mmHg), and in particular ranging from 1.3 Pa to 1300 Pa (0.01 to 10 mmHg).

In addition, the volatile oil generally has a boiling point, measured at atmospheric pressure, ranging from 150° C. to 260° C., and in particular ranging from 170° C. to 250° C.

Advantageously, the volatile oil contains one or more volatile organic oils, the flashpoint of which ranges from 30° C. to 102° C., in particular from 40° C. to 55° C., and especially from 40° C. to 50° C., and mixtures thereof. The term “non-volatile oil” is means any medium capable of remaining on the skin for several hours. A non-volatile oil has in particular, at ambient temperature and atmospheric pressure, a non-zero vapour pressure of less than 0.001 mmHg (0.13 Pa).

The liquid fatty phase (or oily phase) of the composition (which may be in the form of an emulsion) according to the invention preferably contains at least one volatile hydrocarbon-based oil, which, according to a preferred aspect of the invention, is at least isohexadecane.

The term “hydrocarbon-based oil” means an oil essentially made up of, or even being constituted only of, carbon and hydrogen atoms and, optionally, oxygen and nitrogen atoms, and containing no silicon or fluorine atoms. It may contain alcohol, ester, ether, carboxylic acid, amine and/or amide groups.

As volatile oil that can be used in the invention, mention may be made of linear or branched, volatile hydrocarbon-based oils containing from 8 to 16 carbon atoms, and mixtures thereof, and in particular branched C₈-C₁₆ alkanes, such as C₈-C₁₆ isoalkanes (also known as isoparaffins), isododecane, isohexadecane and, for example, the oils sold under the trade names Isopars® or Permethyls®, branched C₈-C₁₆ esters, such as isohexyl neopentanoate, and mixtures thereof. Isohexadecane is in particular used.

The volatile oils may also be silicone oils optionally comprising alkyl or alkoxy groups that are pendent or at the end of a silicone chain, volatile fluoro oils, and mixtures thereof.

As volatile silicone oil that can be used in the invention, mention may be made of linear, branched or cyclic silicone oils having a viscosity at ambient temperature of less than 8 mm²/s and containing, in particular, from 2 to 7 silicon atoms, these silicones optionally comprising alkyl or alkoxy groups containing from 1 to 10 carbon atoms, which are pendent or at the end of each silicone. As volatile silicone oil that can be used in the invention, mention may in particular be made of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

The volatile fluoro oil generally has no flashpoint.

As volatile fluoro oil, mention may be made of nonafluoroethoxybutane, nonafluoromethoxybutane, decafluoropentane, tetradecafluorohexane and dodecafluoropentane, and mixtures thereof.

The amount of additional volatile oil is not limited. For example, it may be present at a content ranging from 1% to 80% by weight, relative to the total weight of the composition, preferably from 2% to 50% by weight, and even more preferably from 3% to 30%.

The oily phase of the composition according to the invention may comprise, in addition to the dipentaerythritol ester, at least one non-volatile oil.

As non-volatile oils that can be used in the invention, mention may be made of hydrocarbon-based oils of mineral or synthetic origin, such as linear or branched hydrocarbons, for instance liquid paraffin or its derivatives, liquid petroleum jelly, polydecenes, hydrogenated polyisobutene such as Parleam sold by the company Nippon Oil Fats, squalane of synthetic or plant origin; oils of animal origin, such as mink oil, turtle oil, perhydrosqualene; hydrocarbon-based oils of plant origin that have a high content of triglycerides made up of fatty acid esters of glycerol, the fatty acids of which may have varied chain lengths, it being possible for the latter to be linear or branched, and saturated or unsaturated, in particular the triglycerides of a fatty acid containing in particular from 4 to 22 carbon atoms, for instance heptanoic acid triglycerides, octanoic acid triglycerides and capric/caprylic acid triglycerides, or else hydroxylated triglycerides, such as sweet almond oil, calophyllum oil, palm oil, grape seed oil, sesame oil, arara oil, rapeseed oil, sunflower oil, cottonseed oil, apricot oil, castor oil, alfalfa oil, marrow oil, blackcurrant oil, macadamia oil, muscat rose oil, hazelnut oil, avocado oil, jojoba oil, olive oil, cereal (maize, wheat, barley, rye) germ oil, shea butter oil; esters of fatty acids, in particular having from 4 to 22 carbon atoms, and in particular of octanoic acid, of heptanoic acid, of lanolic acid, of oleic acid, of lauric acid or of stearic acid, such as propylene glycol dioctanoate, propylene glycol monoisostearate, polyglyceryl-2 diisostearate, neopentyl glycol diheptanoate; synthetic esters of formula R₁COOR₂ in which R₁ represents the residue of a linear or branched higher fatty acid containing from 7 to 40 carbon atoms and R₂ represents a branched hydrocarbon-based chain containing from 3 to 40 carbon atoms, for instance purcellin oil (cetostearyl octanoate), isononyl isononanoate, isodecyl neopentanoate, C₁₂ to C₁₅ alkyl benzoate, 2-ethylhexyl palmitate, 2-octyldodecyl stearate, 2-octyldodecyl erucate, isostearyl isostearate, 2-octyldodecyl benzoate, octanoates, decanoates or ricinoleates of alcohols or of polyalcohols, isopropyl myristate, isopropyl palmitate, butyl stearate, hexyl laurate, diisopropyl adipate, 2-ethylhexyl palmitate, 2-hexyldecyl laurate, 2-octyldecyl palmitate, 2-octyldodecyl myristate, 2-diethylhexyl succinate, diisostearyl malate, glyceryl or diglyceryl triisostearate; hydroxylated esters such as isostearyl lactate, octyl hydroxystearate, octyldodecyl hydroxystearate, diisostearyl malate, triisocetyl citrate; diethylene glycol diisononanoate; pentaerythritol esters; esters of aromatic acids and of alcohols containing from 4 to 22 carbon atoms, in particular tridecyl trimellitate; C₈-C₂₆ higher fatty acids such as oleic acid, linoleic acid, linolenic acid or isostearic acid; C₈-C₂₆ higher fatty alcohols such as oleyl alcohol, linoleyl alcohol, linolenyl alcohol, isostearyl alcohol or octyldodecanol; synthetic ethers containing at least 7 carbon atoms, silicone oils such as linear polydimethylsiloxanes (PDMSs) that are liquid at ambient temperature, and that are optionally phenylated, such as phenyl trimethicones, phenyltrimethylsiloxydiphenylsiloxanes, diphenyl dimethicones, diphenylmethyldiphenyltrisiloxanes, liquid 2-phenylethyltrimethylsiloxysilicates, optionally substituted with aliphatic and/or aromatic groups, for instance alkyl, alkoxy or phenyl groups, which are pendent and/or at the end of a silicone chain, these groups containing from 2 to 24 carbon atoms, and optionally fluorinated, or with functional groups such as hydroxyl, thiol and/or amine groups; polysiloxanes modified with fatty acids or fatty alcohols or polyoxyalkylenes, such as dimethicone copolyols or alkyl methicone copolyols; liquid fluorosilicones; or else caprylic/capric acid triglycerides such as those sold by the company Stearineries Dubois or those sold under the names Miglyol 810, 812 and 818 by the company Dynamit Nobel, and mixtures thereof.

According to a specific embodiment, the non-volatile oil other than the dipentaerythritol ester may be chosen from 2-ethylhexyl palmitate, isodecyl neopentanoate, dicapryl carbonate and 2-octyldodecyl stearate, and a mixture thereof.

The amount of one or more non-volatile oils is not limited. For example, non-volatile oil may be present at a content ranging from 0.1% to 80% by weight, preferably from 1% to 50% by weight, and even more preferably from 2% to 30% by weight, relative to the total weight of the composition.

The oily phase of the composition according to the invention may also comprise one or more of: at least one wax, at least one gum and at least one pasty fatty substance, of plant, animal, mineral or synthetic origin, which may or may not comprise silicone.

For the purpose of the present invention, the term “wax” is intended to mean a lipophilic fatty compound, which is solid at ambient temperature (25° C.), with a reversible solid/liquid change of state, having a melting point of greater than 30° C., which may be up to 200° C., and a hardness of greater than 0.5 MPa, and having in the solid state an anisotropic crystalline organization. By bringing the wax to its melting point, it is possible to make it miscible with the oils and to form a microscopically homogeneous mixture, but on returning the temperature of the mixture to ambient temperature, recrystallization of the wax in the oils of the mixture is obtained.

For the purpose of the present invention, the waxes may be hydrocarbon-based, silicone-based and/or fluoro waxes, optionally comprising ester or hydroxyl functions. They are in particular of natural origin, such as optionally modified beeswax, carnauba wax, candelilla wax, ouricury wax, Japan wax, cork fibre wax, sugarcane wax, ceresin, paraffin wax, lignite wax, microcrystalline waxes, lanolin wax, montan wax, ozokerites, and hydrogenated oils, such as hydrogenated jojoba oil or copolymerization of ethylene, waxes obtained by Fischer-Tropsch synthesis, fatty acid esters and glycerides that are solid at 45° C., silicone waxes, for instance alkyl or alkoxy poly(di)methylsiloxanes and/or polydimethylsiloxane esters that are solid at 45° C., and that contain from 10 to 45 carbon atoms, and certain fatty acids such as stearic acid, myristic acid or behenic acid, and mixtures thereof.

The wax may represent from 0.1% to 30% by weight, in particular from 0.5% to 20% by weight, relative to the total weight of the composition. According to one embodiment, the composition may be free of waxes.

For the purpose of the invention, the term “pasty compound” is intended to mean a compound having a melting point ranging from 25 to 60° C., preferably from 35 to 45° C., and a hardness ranging from 0.001 to 0.5 MPa, preferably from 0.005 to 0.4 MPa.

By way of example of pasty fatty substances, mention may be made of PDMSs with pendent chains of the alkyl or alkoxy type containing from 8 to 24 carbon atoms, such as stearyl dimethicone, and in particular those sold by Dow Corning under the references DC2503 or DC05514; esters of fatty alcohols or of fatty acids containing from 20 to 25 carbon atoms (a melting point in particular of 20 to 35° C. and/or a viscosity at 40° C. ranging from 0.1 to 40 Pa·s), for instance cholesterol esters, such as hydrogenated triglycerides of plant origin, for instance the hydrogenated castor oil sold under the name “Thixin R” by the company Rheox, polyvinyl laurate, arachidyl propionate, triisostearyl citrate or cetyl citrate, PVP/eicosene copolymer; isopropyl lanolins having a viscosity of 10 to 25 Pa·s, preferably of 19 to 25 Pa·s, and/or a melting point of 25 to 45° C., and mixtures thereof.

The composition of the invention may also comprise at least one alkyl, alkoxy or phenyl dimethicone, for instance the product sold under the name “Abil wax 2440®” by the company Goldschmidt.

### Aqueous Phase

The composition according to the invention may comprise at least one aqueous phase comprising water, the amount thereof not being limited.

The water may be of any type, including floral water such as cornflower water and/or a mineral water such as eau de Vittel, eau de Lucas or eau de La Roche Posay and/or a spring water.

This aqueous phase may also comprise organic solvents that are water-miscible (25° C.), for instance primary alcohols such as ethanol and isopropanol, glycols such as glycerol, propylene glycol, butylene glycol, dipropylene glycol, diethylene glycol, glycol ethers, C₁-C₄ alkyl ethers of mono-, di- or tripropylene glycol, or mono-, di- or triethylene glycol, and mixtures thereof.

The aqueous phase may also comprise one or more stabilizing agents, for example sodium chloride, magnesium dichloride and magnesium sulphate.

It may also incorporate any water-soluble or water-dispersible compound compatible with an aqueous phase, such as gelling agents, film-forming polymers, thickeners or surfactants, and mixtures thereof.

According to a specific embodiment, the aqueous phase, and in particular water, may be present in the composition according to the invention at a content ranging from 1% to 60% by weight, in particular from 5% to 50% by weight, relative to the total weight of the composition.

### Surfactants

When the composition according to the invention is in the form of an emulsion comprising an oily continuous phase (W/O), it may comprise at least one emulsifying surfactant.

As emulsifying surfactant, mention may, for example, be made of fatty esters of polyol, in particular of glycerol, of sorbitol or of polyethylene glycol, and in particular polyol isostearates, oleates and ricinoleates such as the mixture of petrolatum, of polyglyceryl-3 oleate, of glyceryl isostearate, hydrogenated castor oil and of ozokerite, sold under the name Protegin W® by the company Goldschmidt, the mixture of glyceryl stearate and of PEG-100 stearate, sold under the name Arlacel 165 by the company ICI, sorbitan isostearate, polyglyceryl diisostearate, polyglyceryl-2 sesquiisostearate; saccharide esters and ethers such as “methyl glucose dioleate”; fatty esters such as magnesium lanolate; dimethicone copolyols and alkyl dimethicone copolyols such as lauryl methicone copolyol sold under the name Dow Corning 5200 Formulation Aid by the company Dow Corning and cetyl dimethicone copolyol sold under the name Abil EM 90® by the company Goldschmidt, the mixture of ethylene glycol acetyl stearate and of glyceryl tristearate sold by the company Guardian under the trade name Unitwix, the copolymer of polyethylene glycol (30 EO) and of 12-hydroxystearic acid sold under the reference Arlacel P 135 by the company Uniqema, and mixtures thereof.

The emulsifying surfactants may be introduced as they are or in the form of a mixture with other emulsifying surfactants and/or with other compounds such as fatty alcohols or oils.

As emulsifiers, use may also be made of amphiphilic polymers such as modified acrylic copolymers, for instance the products sold under the name Pemulen by the company Goodrich; copolymers of 2-acrylamido-2-methylpropanesulphonic acid containing a hydrophobic chain, as described in document EP-A-1 069 142; polyolefins containing an optionally esterified or amidated succinic end group, for instance the compounds described in documents U.S. Pat. No. 4,234,435, U.S. Pat. No. 4,708,753, U.S. Pat. No. 5,129,972, U.S. Pat. No. 4,931,110, GB-A-2,156,799 and U.S. Pat. No. 4,919,179. As polyolefins containing a succinic end group, mention may in particular be made of polyisobutylenes containing a modified succinic end group, such as the products sold under the names L2724 and L2721 by the company Lubrizol.

The amount of emulsifying surfactant can range from 0.1% to 10% by weight, relative to the total weight of the composition, and in particular from 1% to 5% by weight.

### Thickener

The emulsion according to the invention may also comprise one or more agents for thickening the fatty phase. The thickening agent may be chosen from:

organomodified clays, which are clays treated with compounds chosen in particular from quaternary amines and tertiary amines. As organomodified clays, mention may be made of organomodified bentonites such as those sold under the name “Bentone 34” by the company Rheox, organomodified hectorites such as those sold under the name “Bentone 27” and “Bentone 38” by the company Rheox,

hydrophobic fumed silica, which is a fumed silica that has been chemically surface-modified by means of a chemical reaction generating a decrease in the number of silanol groups. Silanol groups can in particular be substituted with hydrophobic groups.

The hydrophobic groups may be:

trimethylsiloxyl groups, which are in particular obtained by treating fumed silica in the presence of hexamethyldisilazane. Silicas thus treated are called “Silica silylate” according to the CTFA (6th edition, 1995). They are, for example, sold under the references “Aerosil R812®” by the company Degussa and “CAB-O-SIL TS-530®” by the company Cabot,

dimethylsilyloxyl or polydimethylsiloxane groups, which are in particular obtained by treating fumed silica in the presence of polydimethylsiloxane or of dimethyldichlorosilane. Silicas thus treated are called “Silica dimethyl silylate” according to the CTFA (6th edition, 1995). They are, for example, sold under the references “Aerosil R972®”, and “Aerosil R974®” by the company Degussa, and “CAB-O-SIL TS610” and “CAB-O-SIL TS-720®” by the company Cabot.

The fumed silica preferably has a particle size that may be nanometric to micrometric, for example ranging approximately from 5 to 200 nm.

The agent for thickening the fatty phase may be present at a content ranging from 0.1% to 10% by weight, and in particular from 1% to 5% by weight, relative to the total weight of the composition.

### Particulate Phase

The particulate phase of the composition according to the invention may also comprise at least one additional filler different from the particles of acrylic polymer.

The term “filler” is intended to mean any colourless particle chosen from mineral or organic fillers which are lamellar, spherical or oblong, and which are chemically inert in the composition.

Mention may be made of talc, mica, silica, kaolin, polyamide, such as Nylon®, powders, poly-β-alanine and polyethylene powders, powders of tetrafluoroethylene (Teflon®) polymers, lauroyl lysine, starch, boron nitride, polymeric hollow microspheres such as those made of polyvinylidene chloride/acrylonitrile, for instance Expancel® (Nobel Industrie), polyurethane powders, silicone resin microbeads (Tospearls® from Toshiba, for example), precipitated calcium carbonate, dicalcium phosphate, magnesium carbonate, magnesium hydrogen carbonate, hydroxyapatite, hollow silica microspheres (Silica Beads® from Maprecos), glass or ceramic microcapsules, metal soaps derived from organic carboxylic acids containing from 8 to 22 carbon atoms, preferably from 12 to 18 carbon atoms, for example zinc stearate, magnesium stearate or lithium stearate, zinc laurate, magnesium myristate, and mixtures thereof. These fillers may or may not be surface-treated, in particular to make them lipophilic.

The fillers may be present in the composition according to the invention at a content ranging from 0.1% to 30% by weight, preferably from 0.5% to 20% by weight, and even more preferably from 1% to 10%, relative to the total weight of the composition.

### Dyestuffs

The composition may advantageously contain at least one dyestuff which may be chosen from the lipophilic dyes, the hydrophilic dyes, the pigments and the pearlescent agents normally used in cosmetic or dermatological compositions, and mixtures thereof. This dyestuff is generally present in a proportion of from 0.001% to 40% by weight, especially from 0.1% to 30% by weight, in particular from 2% to 25% by weight, and more particularly from 3% to 20% by weight, relative to the total weight of the composition.

The synthetic or natural lipophilic dyes may, for example, be chosen from DC Red 17, DC Red 21, DC Red 27, DC Green 6, DC Yellow 11, DC Violet 2, DC Orange 5, Sudan Red, carotenes (P-carotene, lycopene), xanthophylls (capsanthin, capsorubin, lutein), palm oil, Sudan Brown, Quinoline Yellow, annatto and curcumin.

The synthetic or natural water-soluble dyes may, for example, be chosen from FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper chlorophyllin, methylene blue, anthocyanins (enocyanin, black carrot, hibiscus, elder), caramel and riboflavin.

The dyes may also be chosen from juglone, lawsone, extracts of fermented soybean, algal extracts, fungal extracts, extracts of microorganisms, flavylium salts unsubstituted in the 3-position, such as, for example, those described in patent EP 1 172 091, extracts of Gesneria fulgens, Blechum procerum, or Saxifraga, and pigments that can be obtained by extraction with an organic or aqueous-organic solvent of a culture medium of micromycetes of the Monascus type.

The dyes may represent from 0.001% to 3% by weight, and in particular from 0.01% to 1% by weight, relative to the total weight of the composition.

The pigments may be white or coloured, mineral and/or organic, and coated or uncoated. Among mineral pigments, mention may be made of optionally surface-treated titanium dioxide, zirconium oxide or cerium oxide and also iron oxide or chromium oxide, manganese violet, ultramarine blue, chromium hydrate and ferric blue. Among organic pigments, mention may be made of carbon black, pigments of D&C type, and lakes based on cochineal carmine, on barium, on strontium, on calcium or on aluminium. The pigments may represent from 0.1% to 40% by weight, especially from 1% to 35% by weight, and in particular from 2% to 25% by weight, relative to the total weight of the composition.

The pearlescent agents may be chosen from white pearlescent pigments such as mica coated with titanium or with bismuth oxychloride, coloured pearlescent pigments such as titanium mica with iron oxides, titanium mica with, in particular, ferric blue or chromium oxide, titanium mica with an organic pigment of the abovementioned type, and also pearlescent pigments based on bismuth oxychloride. They may represent from 0.1% to 20% by weight, and in particular from 0.1% to 15% by weight, relative to the total weight of the composition.

Advantageously, the emulsion according to the invention comprises hydrophobic coated pigments. The hydrophobic coated pigments are pigments (such as those mentioned above) surface-treated with a hydrophobic agent in order to make them compatible with the fatty phase of the emulsion, in particular so that they have good wettability with the oils of the fatty phase such that these treated pigments are well dispersed in the fatty phase.

The hydrophobic treatment agent may be chosen from silicones such as methicones, dimethicones, perfluoroalkylsilanes; fatty acids such as stearic acid; metal soaps such as aluminium dimyristate, the aluminium salt of hydrogenated tallow glutamate, perfluoroalkyl phosphates, perfluoroalkylsilanes, perfluoroalkylsilazanes, poly(hexafluoropropylene oxide)s, polyorganosiloxanes comprising perfluoroalkyl perfluoropolyether groups, amino acids; N-acylated amino acids or salts thereof; lecithin, isopropyl triisostearyl titanate, and mixtures thereof.

The N-acylated amino acids may comprise an acyl group containing from 8 to 22 carbon atoms, for instance a 2-ethylhexanoyl, caproyl, lauroyl, myristoyl, palmitoyl, stearoyl or cocoyl group. The salts of these compounds may be the aluminium, magnesium, calcium, zirconium, zinc, sodium or potassium salts. The amino acid may, for example, be lysine, glutamic acid or alanine.

The term “alkyl” mentioned in the compounds which are hydrophobic agents, mentioned above, denotes in particular an alkyl group containing from 1 to 30 carbon atoms, preferably containing from 5 to 16 carbon atoms.

Hydrophobic-treated pigments are in particular described in application EP-A-1086683.

### Additives

The composition of the invention may also comprise any additional additive such as antioxidants, preservatives, neutralizing agents, lipophilic gelling agents, aqueous-phase gelling agents, dispersing agents, active cosmetic agents, fragrances, free-radical scavengers, sequestering agents and film-forming agents, and mixtures thereof. These additives may be present in the composition in a proportion of from 0.0005% to 20% by weight, and in particular from 0.001% to 10% by weight, relative to the total weight of the composition.

As active cosmetic agent that can be used in the invention, mention may be made of vitamins A, E, C, B₃ and F, provitamins such as D-panthenol, calmative active agents such as α-bisabolol, aloe vera, allantoin, plant extracts or essential oils, protective or restructuring agents such as ceramides, “freshening” active agents such as menthol and its derivatives, emollients, moisturizers, active antiwrinkle agents, essential fatty acids and sunscreens, and mixtures thereof.

Of course, those skilled in the art will take care to select the optional additional additives and/or the amount thereof in such a way that the advantageous properties of the composition according to the invention are not, or are not substantially, impaired by the addition envisaged.

The composition according to the invention may be produced by one of ordinary skill in the art using known methods, for example those generally used in the cosmetics field, without burden in view of this disclosure.

The composition according to the invention has an oily continuous phase, and may in particular be in the form of an anhydrous product or of a water-in-oil (W/O) emulsion or else an oil-in-water-in-oil triple emulsion (O/W/O). According to a preferred embodiment, the composition is in the form of a W/O emulsion.

The composition according to the invention may be a composition for making up the skin, such as a foundation, a concealer product or a tinted cream, or a composition for making up the body, and in particular a foundation to be applied to the face and/or the neck.

The invention is illustrated in greater detail in the examples below.

### EXAMPLES

The examples given below are presented by way of nonlimiting illustration of the field of the invention.

### Examples 1 to 10 of Foundations

The following foundation compositions were prepared:

| | | % by |
|---|---|---|
| | | weight |
| A1 | Copolymer of polyethylene glycol (30 EO) and of | 4.00 |
| | 12-hydroxystearic acid sold under the reference | |
| | Arlacel P 135 by the company Uniqema | |
| | 2-Ethylhexyl palmitate | 2.00 |
| | Mixture of glyceryl tristearate and of acetylated | 0.70 |
| | ethylene glycol stearate sold under the reference | |
| | Unitwix by the company United Guardian | |
| | Dicapryl carbonate | 3.60 |
| | 2-Octyldodecyl stearate | 4.00 |
| | 2-Ethylhexyl para-methoxycinnamate | 5.00 |
| | Isohexadecane | 3.60 |
| | OIL | 10.00 |
| | Preservative | 0.15 |
| A2 | Bentone gel sold under the reference Bentone Gel | 4.00 |
| | IHD V by the company Elementis | |
| A3 | Isodecyl neopentanoate | 4.20 |
| | Yellow iron oxide coated with aluminium stearoyl | 1.80 |
| | glutamate | |
| | Brown iron oxide coated with aluminium stearoyl | 0.49 |
| | glutamate | |
| | Black iron oxide coated with aluminium stearoyl | 0.21 |
| | glutamate | |
| | Titanium dioxide coated with aluminium stearoyl | 7.50 |
| | glutamate | |
| A4 | FILLER | 5.00 |
| B | Demineralized water | 37.15 |
| | Preservative | 0.40 |
| | Magnesium sulphate | 0.60 |
| | EDTA | 0.10 |
| | Octane-1,2-diol | 0.50 |
| | Glycerol | 5.00 |
| | TOTAL | 100% |

### Protocol

The constituents of phase A1 are weighed into the main beaker and the latter is placed in a water bath (85° C.).

When the mixture is homogeneous, A2 is added with stirring using a Moritz stirrer at 1500 rpm.

Phase A3 is prepared separately by milling the pigments with a three-roller mill, and is then added to the above mixture, with stirring.

Phase A4 is then incorporated while maintaining the same stirring.

To prepare aqueous phase B, the water, the glycerol and the preservative are weighed out and are brought to boiling until complete dissolution is obtained. The magnesium sulphate and the EDTA are then incorporated, followed, finally, by the octane-1,2-diol at 45° C.

The emulsion is prepared at ambient temperature: the aqueous phase is poured into the fatty phase, gradually increasing the stirring speed of the Moritz stirrer to 4000-4500 rpm.

After addition of the aqueous phase, the mixture is left to stir for a further 10 min.

### Influence of the Nature of the Filler:

The influence of the nature of the filler on the properties of the foundations according to the invention was compared. For this, the dipentaerythrityl hexacaprylate/caprate sold under the name DUB DPHCC by the company Stearinerie Dubois was chosen as oil in the composition exemplified above.

The fillers tested are the following:

| | FILLER |
|---|---|
| EX1 | Methyl methacrylate/ethylene glycol dimeth- |
| (invention) | acrylate copolymer sold under the reference |
| | Techpolymer MBP-8 by the company Sekisui |
| | Plastics |
| EX2 | Nylon 12 sold under the reference Orgasol |
| (comparison) | 2002 EXD NAT COS by the company Arkema |
| EX3 | Polymethylsilsesquioxane sold under the |
| (comparison) | reference Tospearl 145 A by the company |
| | GE Toshiba Silicones |
| EX4 | Silica sold under the reference Sunsphere H51 |
| (comparison) | by the company Asahi Glass SI-Tech |

The cosmetic properties of the compositions obtained were compared by a panel of 5 experts.

For each test, half the face was made up with an amount of 0.15 g of foundation according to Example 1 (invention) and the other half of the face was made up with a composition according to Examples 2, 3 or 4 (not part of the invention).

Example 1 (according to the invention), in comparison with the foundation according to Example 2 (not part of the invention), forms a film that is less tacky once applied to the skin.

In comparison with Examples 3 and 4 (not part of the invention), the foundation according to Example 1 (invention) gives a less greasy make-up result.

### Influence of the Nature of the Oil:

The influence of the nature of the oil on the properties of the foundations according to the invention was compared. For this, the methyl methacrylate/ethylene glycol bismethacrylate copolymer sold under the reference Techpolymer MBP-8 by the company Sekisui Plastics was used as filler in the composition exemplified above.

The oils tested are the following:

| | OIL |
|---|---|
| EX1 | Dipentaerythrityl hexacaprylate/caprate sold |
| (invention) | under the reference DUB DPHCC by the company |
| | Stearinerie Dubois |
| EX5 | Pentaerythrityl tetra caprylate/caprate sold |
| (comparison) | under the reference DUB PTCC by the company |
| | Stearinerie Dubois |
| EX6 | Pentaerythrityl tetra(2-ethylhexanoate) sold |
| (comparison) | under the reference Trivent PE 48 by the |
| | company Alzo |
| EX7 | Pentaerythrityl tetra(2-hexyldecanoate) sold |
| (comparison) | under the reference Isocarb ester 1605 by the |
| | company Sasol |
| EX8 | Pentaerythrityl tetraisostearate sold under the |
| (comparison) | reference Prisorine 3631 by the company Uniqema |
| EX9 | Glyceryl triisostearate sold under the |
| (comparison) | reference DUB TGIS by the company Stearinerie |
| | Dubois |
| EX10 | Glyceryl tri(2-decyltetradecanoate) sold under |
| (comparison) | the reference DUB TGI 24 by the company |
| | Stearinerie Dubois |

The cosmetic properties of the compositions obtained were compared by a panel of 5 experts.

For each test, half the face was made up with an amount of 0.15 g of foundation according to Example 1 (invention) and the other half of the face was made up with a composition according to Examples 5 to 10 (not part of the invention).

The foundation according to the Example 1 (invention), compared with the foundations according to Examples 5 to 10 (not part of the invention), forms a film that is less greasy once applied to the skin. The make-up finish is more powdery.

In comparison with Examples 5 and 9 (not part of the invention), Example 1 makes it possible to form a make-up film that is less tacky once applied to the skin.

### Example 11 of a Foundation

A foundation having the following composition was prepared:

| | | % by |
|---|---|---|
| | | weight |
| A1 | Copolymer of polyethylene glycol (30 EO) and of | 4.00 |
| | 12-hydroxystearic acid sold under the reference | |
| | Arlacel P 135 by the company Uniqema | |
| | 2-Ethylhexyl palmitate | 2.80 |
| | Mixture of glyceryl tristearate and of acetylated | 0.70 |
| | ethylene glycol stearate sold under the reference | |
| | Unitwix by the company United Guardian | |
| | Dicapryl carbonate | 5.00 |
| | 2-Octyldodecyl stearate | 5.60 |
| | 2-Ethylhexyl para-methoxycinnamate | 5.00 |
| | Isohexadecane | 5.00 |
| | Dipentaerythrityl hexa C5-9 carboxylate sold under | 3.00 |
| | the name LexFeel 350 by the company Inolex | |
| | Chemical Company | |
| | Preservative | 0.15 |
| A2 | Bentone gel sold under the reference Bentone Gel | 4.00 |
| | IHD V by the company Elementis | |
| A3 | Isodecyl neopentanoate | 6.00 |
| | Yellow iron oxide coated with aluminium stearoyl | 1.80 |
| | glutamate | |
| | Brown iron oxide coated with aluminium stearoyl | 0.49 |
| | glutamate | |
| | Black iron oxide coated with aluminium stearoyl | 0.21 |
| | glutamate | |
| | Titanium dioxide coated with aluminium stearoyl | 7.50 |
| | glutamate | |
| A4 | Methyl methacrylate/ethylene glycol dimethacrylate | 5.00 |
| | copolymer sold under the reference Techpolymer | |
| | MBP-8 by the company Sekisui Plastics | |
| B | Demineralized water | 37.15 |
| | Preservative | 0.40 |
| | Magnesium sulphate | 0.60 |
| | EDTA | 0.10 |
| | Octane-1,2-diol | 0.50 |
| | Glycerol | 5.00 |
| | TOTAL | 100% |

The protocol is the same as that for Examples 1 to 10.

A foundation is obtained which has good softness and spreading properties when applied to the skin and which does not leave a greasy or even tacky feeling on application or during the day.

The above written description of the invention provides a manner and process of making and using it such that any person skilled in this art is enabled to make and use the same, this enablement being provided in particular for the subject matter of the appended claims, which make up a part of the original description and including a composition for, e.g., making up or caring for the skin, comprising an oily continuous phase and a particulate phase, said oily continuous phase comprising at least one dipentaerythritol ester obtained by complete esterification of dipentaerythritol with linear or branched, saturated or unsaturated acids containing from 3 to 24 carbon atoms, preferably from 4 to 18 carbon atoms, and even more preferably from 5 to 16 carbon atoms, and said particulate phase comprising particles of acrylic polymer.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition comprising an oily continuous phase and a particulate phase, wherein said oily continuous phase comprises at least one dipentaerythritol C₃₋₂₄ester, and wherein said particulate phase comprises particles of acrylic polymer.

2. The composition according to claim 1, wherein the particles of acrylic polymer are not film-forming.

3. The composition according to claim 1, wherein the particles of acrylic polymer are chosen from:
particles of crosslinked or noncrosslinked polymethyl methacrylate,
particles of methyl methacrylate/butyl acrylate copolymer,
particles of methyl acrylate/ethylene copolymer,
particles of methyl methacrylate/ethylene glycol dimethacrylate crosslinked copolymer,
particles of polymethyl methacrylate/ethylene glycol dimethacrylate copolymer,
particles of methyl methacrylate/ethylene glycol dimethacrylate crosslinked copolymer,
particles of polyallyl methacrylate/ethylene glycol dimethacrylate copolymer, and
particles of lauryl methacrylate/ethylene glycol dimethacrylate copolymer.

4. The composition according to claim 1, wherein the particles of acrylic polymer are present in an amount of 0.1% to 10% by weight, relative to the total weight of the composition.

5. The composition according to claim 1, comprising dipentaerythrityl hexacaprylate/caprate.

6. The composition according to claim 1, wherein the dipentaerythritol C₃₋₂₄ester is present in an amount of 0.1% to 30% by weight, relative to the total weight of the composition.

7. The composition according to claim 1, wherein the oily phase comprises at least one volatile oil.

8. The composition according to claim 1, wherein the oily phase comprises isohexadecane at a content ranging from 1% to 80% by weight, relative to the total weight of the composition.

9. The composition according to claim 1, wherein the oily phase comprises at least one non-volatile oil chosen from 2-ethylhexyl palmitate, isodecyl neopentanoate and dicapryl carbonate, and mixtures thereof.

10. The composition according to claim 1, wherein the oily phase comprises at least one non-volatile oil in a content ranging from 0.1% to 80% by weight relative to the total weight of the composition.

11. The composition according to claim 1, further comprising an aqueous phase comprising water in a content of 1% to 60% by weight relative to the total weight of the composition.

12. The composition according to claim 1, further comprising at least one material chosen from lipophilic dyes, hydrophilic dyes, pigments and pearlescent agents, and mixtures thereof, being present in a proportion of from 0.001% to 40% by weight relative to the total weight of the composition.

13. The composition according to claim 1, wherein said composition is in the form of a W/O emulsion.

14. The composition according to claim 1, wherein said dipentaerythritol C₃₋₂₄ester is obtained by complete esterification of dipentaerythritol with linear or branched, saturated or unsaturated acids containing from 3 to 24 carbon atoms.

15. The composition according to claim 1, wherein the oily phase comprises at least one non-volatile oil.

16. The composition according to claim 1, wherein said oily phase comprises at least one dipentaerythritol C₄₋₈ester.

17. The composition according to claim 1, wherein said oily phase comprises at least one dipentaerythritol C₅₋₁₆ester.

18. The composition according to claim 1, wherein:
the composition is in the form of a W/O emulsion,
the particles of acrylic polymer are spherical particles having a number-average size of 3 to 20 microns present in an amount of 0.1% to 10% by weight, relative to the total weight of the composition, and
the composition comprises at least one dipentaerythritol C₅₋₁₆ester in am amount of 0.1% to 30% by weight, relative to the total weight of the composition.

19. The composition according to claim 1, wherein said composition is anhydrous.

20. A process for making up or caring for the skin, comprising applying the composition of claim 1 to the skin.
